(19) United States
(12) Patent Application Publication
(10) Pub. No.: US 2013/0226296 A1
(43) Pub. Date: Aug. 29, 2013

(54) METHODS AND APPARATUS FOR IN SITU FORMATION OF SURGICAL IMPLANTS

(71) Applicant: TransMed7, LLC, (US)

(72) Inventors: Ary S. CHERNOMORSKY, Walnut Creek, CA (US); John K. SOO HOO, Union City, CA (US); James W. VETTER, Portola Valley, CA (US)

(73) Assignee: TransMed7, LLC, Portola Valley, CA (US)

(21) Appl. No.: 13/853,933

(22) Filed: Mar. 29, 2013

Related U.S. Application Data
(62) Division of application No. 13/110,480, filed on May 18, 2011.
(60) Provisional application No. 61/346,326, filed on May 19, 2010.

(51) Int. Cl. A61F 2/12

(52) U.S. Cl. CPC A61F 2/12
USPC 623/8

(57) ABSTRACT

Methods, devices and systems for in situ formation of an implant within a post-surgical cavity. A balloon is provided within the cavity and a gelling initiator such as a cross-linking agent is introduced into the balloon. A polymer susceptible to solidifying in the presence of the gelling initiator is then introduced into the balloon. The introduced polymer is allowed solidify through contact with the introduced gelling initiator to form the implant while the balloon isolates the solidifying implant from the cavity. The balloon is then ruptured and extracted from the cavity such that the formed implant remains within and directly contacts an interior surface of the cavity.

### CROSS-REFERENCE TO RELATED APPLICATIONS

The present application claims priority to copending and commonly assigned U.S. provisional application Ser. No. 61/346,326 filed on May 19, 2010, which application is incorporated herewith by reference.

### BACKGROUND OF THE INVENTION

1. Field of the Invention

Embodiments of the present inventions relate to post-surgical implants and delivery devices and systems for delivering such implants to a post-surgical cavity, such as a post-lumpectomy cavity.

### SUMMARY OF THE INVENTION

According to embodiments thereof the present inventions are drawn to post-surgical implants and methods of forming the same from one or more polymerizing biomaterials that are introduced within a post-surgical cavity in a patient, and controllably solidified to form the implant in situ within the cavity.

According to an embodiment thereof, the present invention is a soft tissue implant formed in situ.

According to another embodiment, the present invention is a method of forming an implant within a post-surgical cavity. The method may include steps of providing a balloon within the cavity; introducing a gelling initiator into the balloon; introducing, into the balloon, a polymer susceptible to solidifying when in contact with the gelling initiator; enabling the introduced polymer to solidify through contact with the introduced gelling initiator to form the implant, and rupturing the balloon and extracting the ruptured balloon from the cavity such that the formed implant remains within and directly contacts an interior surface of the cavity.

According to further embodiments, the polymer and the gelling initiator may be introduced into the balloon separately from one another. The polymer introducing step may be carried out with the polymer including alginate. The gelling initiator providing step may be carried out with the gelling initiator including divalent cations of bivalent metals. The gelling initiator providing step may be carried out with the gelling initiator including a cross-linking agent. The polymer may be introduced into the balloon prior to introducing the gelling initiator into the balloon. Alternatively, the gelling initiator may be introduced into the balloon prior to introducing the polymer into the balloon. The polymer introducing step may be carried out with the polymer including alginate dispersed in an aqueous solution at a concentration of about 0.1% to about 80% by weight. The method may further include a step of expanding the balloon within the cavity. The polymer introducing step may include introducing a volume of about 0.01 cc to about 600 cc of the polymer into the balloon. The gelling initiator introducing step may be carried out by introducing a volume of about 0.01 cc to about 900 cc of the gelling initiator into the balloon. The gelling initiator introducing step may be carried out by introducing the gelling initiator along with a biologically active substance into the balloon. The polymer introducing step may be carried out by introducing the polymer along with a biologically active substance into the balloon. The gelling introducing step may be carried out with the gelling initiator having a predetermined porosity. The gelling initiator introducing step may be carried out with the gelling initiator being configured with divalent cations of bivalent metals coupled with a biologically active substance. The gelling initiator introducing step may be carried out with the gelling initiator having a porosity that is different from a porosity of the polymer. The method may further include a step of foaming the gelling initiator such that the foamed gelling initiator has, exhibits or defines a predetermined porosity. The method may also include a step of introducing gas bubbles into the gelling initiator such that the foamed gelling initiator has, exhibits or defines a predetermined porosity.

The balloon providing step may be carried out with the balloon being configured to isolate the introduced polymer and gelling initiator from bodily fluids within the cavity. The balloon providing step may be carried out with the balloon being configured with locally thinner portions. The balloon providing step may be carried out with the balloon being configured to selectively rupture within the cavity. The balloon rupturing and extracting step may be carried out by pulling the balloon in a proximal direction while the balloon is disposed within the cavity. The balloon extracting step may include causing the ruptured balloon to slide over the framed implant, bringing the formed implant and the cavity into contact. The balloon providing step may be carried out with an interior surface of the balloon further including or defining a porous layer or portion. The method may also include a step or steps of determining relative amounts and concentrations of gelling initiator and polymer to form an implant having desired characteristics in controlled manner.

According to yet another embodiment thereof, the present invention is a delivery system. The delivery system may include a first source of polymer; a second source, separate from the first source, of a gelling initiator that may be configured to gel the polymer, and a catheter configured to deliver the polymer and the gelling initiator to a cavity within the patient such that the delivered polymer and gelling initiator may be initially isolated from the cavity and only selectively exposed to the cavity after the polymer has at least partially gelled.

The catheter may include a balloon configured to isolate the delivered polymer and gelling initiator from the cavity. The balloon may include or otherwise define a porous interior surface, section or portion configured to contain at least a portion of the gelling initiator. The porous interior surface or portion may be configured to release the contained gelling initiator at least when the introduced polymer applies pressure there against.

The polymer may include, for example, alginate. The gelling initiator may include divalent cations. The gelling initiator may include a cross-linking agent. The catheter may be configured to deliver the polymer and the gelling initiator separately to the cavity and to isolate them from the cavity (i.e., from the tissue sidewalls of the cavity) until the polymer has at least partially gelled. The balloon may include locally weaker portions. The delivery system may further include a marker configured to be delivered within the balloon within the cavity.

### BRIEF DESCRIPTION OF THE DRAWINGS

FIG. 1 is a flowchart of a method for the formulation and formation of a post-surgical implant, according to an embodiment of the present inventions.

FIG. 2A shows illustrations of a breast, breast tumor, postsurgical cavity, and dissected tissue.

FIG. 2B shows a delivery system according to an embodiment of the present inventions.

FIG. 3A shows the delivery device according to embodiments of the present inventions, inserted into a trocar introducer that has pierced the breast tissue and has been introduced in proximity to the postsurgical cavity. FIG. 3B shows the inflated balloon in an expanded or inflated state within the cavity, according to embodiments of the present inventions.

FIG. 4A shows the delivery system according to embodiments of the present invention, in which the balloon is in an inflated state and in which the implant is being formulated/formed.

FIG. 4B shows the delivery system according to embodiments of the present invention, in which the implant is being formed through continued injection of the polymer into the catalyst or gelling initiator, gradually displacing and becoming bathed in the gelling initiator, thereby enabling a reaction between the polymer and the gelling initiator to form a self-polymerizing solidified or solidifying material within the balloon disposed within the cavity.

FIG. 4C shows the delivery system according to embodiments of the present invention, in which continued injection of the polymer into the gelling initiator fills the interior of the balloon and enables a reaction between the polymer and the gelling initiator to form a self-polymerizing solidified or solidifying implant within the interior of the balloon, which is itself disposed within the post-surgical cavity.

FIG. 4D shows the solidified or solidifying implant formed within the balloon and the process of rupturing the balloon, for the consequent removal of the ruptured balloon from the cavity, leaving the resident implant at least partially conformed to the surrounding cavity interior, according to embodiments of the present inventions.

FIG. 4E shows the solidified or solidifying implant formed within the balloon and the ruptured balloon being extracted from the cavity, leaving the resident implant conformed to the surrounding cavity interior, according to embodiments of the present inventions.

FIG. 5A shows a cross-sectional side view of an embodiment of the present delivery system, showing the outer surface of the balloon, the inner porous surface thereof, as well as the lumen(s) for the delivery of the polymer and/or the gelling initiator, according to embodiments of the present inventions.

FIG. 5B shows a cross-sectional side view of an embodiment of the present delivery system, showing the balloon inflated with the gelling initiator or catalyst and having a porous inner surface saturated with the catalyst, according to embodiments of the present inventions.

FIG. 6A shows further aspects of the in situ formation of an implant within a balloon, according to embodiments of the present inventions.

FIG. 6B shows further aspects of the gradual formation of a solidified outer implant layer at the points of contact where the introduced polymer contacts the gelling initiator or catalyst, according to embodiments of the present inventions.

FIG. 7A shows aspects of an embodiment of a delivery device in which the balloon thereof has one or more notches formed near or at a distal end of the balloon, according to embodiments of the present inventions.

FIG. 7B shows aspects of the extraction of the balloon from the cavity, leaving the solidified or solidifying implant in place within the cavity, according to embodiments of the present inventions.

### DETAILED DESCRIPTION OF THE INVENTION

Many medical procedures require the surgical formation and maintenance of a cavity within a patient's body. For example, the treatment of certain tumors may require a multi-faceted approach that includes a combination of surgery, radiation therapy and chemotherapy. In such an approach, after an initial surgical procedure has been performed to remove as much of a tumor as possible, radiation and chemotherapy are performed to kill remaining cancerous cells that could not be removed surgically.

More than 1,250,000 reconstructive procedures are performed on the breast each year. Surgically formed cavities, particularly post-lumpectomy cavities, often cause local deformation of the tissue surrounding the lumpectomy site, leading to poor cosmetic results. Women afflicted with breast cancer, congenital defects or damage resulting from trauma typically have very few alternatives to breast reconstruction. Breast reconstruction is frequently performed at the time, or shortly after, mastectomy or lumpectomy for cancer treatments. Reconstructive procedures frequently involve moving vascularized skin flaps with underlying connective and adipose tissue from one region of the body, e.g., the buttocks or the abdominal region, to the breast region, resulting in additional trauma to the patient and longer healing times. Often, surgeons use synthetic breast implants and tissue expanders for reconstruction, which typically require additional procedures and which may cause further complications especially in the patients who went through local radiation therapy such as partial breast irradiation brachytherapy.

Embodiments of the present inventions provide methods, systems and devices for the formulation and formation of biodegradable implants within a surgical resection cavity formed from solid tissue, such as a lumpectomy cavity formed in a breast following breast cancer surgery or other procedure. Such implants may initially be formulated and formed within a containment structure such as a balloon that has been inserted within the cavity in a minimally invasive manner. The balloon may then be expanded and a self-polymerizing biomaterial or combination of biocompatible materials may be selectively introduced therein in a controlled fashion to completely or at least partially fill the interior volume of the balloon. One or more of the biocompatible materials may already be present within the balloon. When the implant has at least partially polymerized (e.g., solidified), the balloon may then be withdrawn from the cavity, leaving the at least partially solidified implant in place, leaving only a small wound for closure.

The formulation and formation of the present implants may be carried out under a variety of guiding visualization modalities, such as ultrasound, for example. Indeed, a small diameter trocar, delivery catheter, and biocompatible biopolymer that is capable of controllable solidification may be selectively introduced into the post-surgical cavity under ultrasonic guidance. Ultrasound and/or other visualization modalities may be used to aid in the planning of immediate or subsequent treatment of the postsurgical cavity.

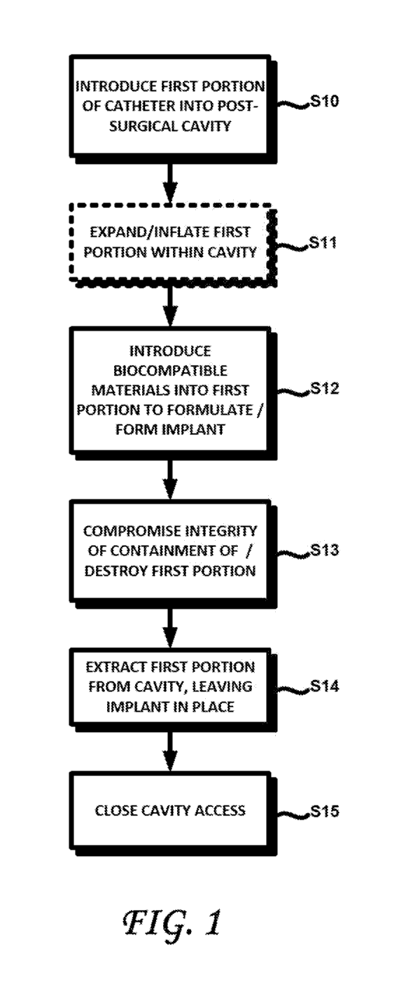

FIG. 1 shows an embodiment of the present methods for the formulation and formation of an implant within a post-surgical cavity. A delivery system such as a catheter may include a first distal portion and a second proximal portion. The first portion may be configured for introduction into the cavity, such as a cavity formed within a breast. The second portion of the catheter may be configured to remain outside of the cavity. According to embodiments of the present inventions, the first portion may include a variable geometry containment structure such as, for example, an inflatable balloon. The second portion may include structures enabling the physician to at least selectively introduce biocompatible materials into the first distal portion of the delivery system. The first distal portion of the delivery system may be inserted within the cavity, as shown at step S10 of FIG. 1. As shown at step S11 the containment structure may then be expanded within the cavity, such as by, for example, inflating the balloon within the cavity or by mechanically expanding the structure. S11, shown in dashed lines in FIG. 1, may be an optional step, as the introduction of the biocompatible materials within the containment structure may act to expand the structure, without the need for a separate expanding or inflating step. One or more biocompatible materials may then be introduced into the first distal portion, such as within the inflated balloon, as called for by step S12. The biocompatible implant then forms of at least some of the introduced biocompatible materials. Others of the introduced (or already present) biocompatible materials may serve a beneficial purpose other than the formation of the implant. A marker may be introduced into the first portion of the catheter, concurrently with one or more of the constituent components of the implant, or before or after the introduction of such constituent components of the implant into the first portion of the catheter. After the formulation and formation of the implant within the containment structure, the containment structure of the first distal portion of the delivery system may then be opened, punctured, breached or otherwise compromised as shown at S13, thereby enabling the containment structure to be safely extracted from the cavity as shown at S14, leaving the just-formed biocompatible implant in place, within the cavity. The cavity may then be closed as shown at S15, although the closing of the access path to the cavity, strictly speaking, need not form part of the present inventions.

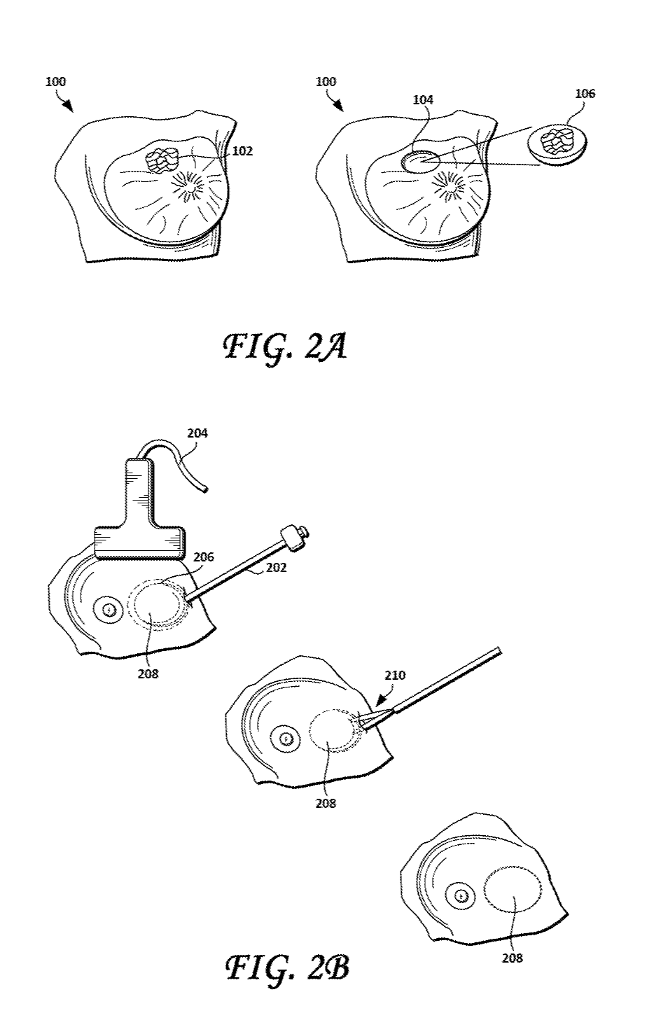

FIG. 2A is an illustration of a breast 100, a breast tumor 102, postsurgical cavity 104 (although shown as an open wound for illustration purposes, the cavity is often located within and surrounded by other breast tissue and accessed through a small opening in the patient's skin), and dissected tissue 106. In sequence, these drawings illustrate a typical lumpectomy procedure that excises a lump (lesion, tumor, sample or specimen) of tissue 106 and leaves behind a postsurgical cavity 104. Such a cavity 104 may cause cosmetically undesirable dimpling, distortion or other deformation of the breast.

FIG. 2B shows a delivery system in which a first distal portion of an implant delivery and formulation device such as a catheter 202 is inserted into a tissue cavity (such as within the breast, for example), under ultrasonic guidance, as suggested at 204. A containment structure of the first distal portion, such as an inflatable balloon 206, may then optionally be expanded or inflated to fill or substantially fill the cavity within the breast. The balloon or other variable geometry containment structure may be expanded or inflated to assume somewhat greater dimensions than the dimensions of the cavity. One or more biocompatible materials may then be introduced within the inflated balloon, to both formulate and form the implant 208 within the inflated balloon. It is to be noted that one or more biologically compatible materials may already be present within the balloon 206 prior to insertion thereof into the cavity. In this manner, the implant 208 may then be formulated (the constituent elements thereof, at predetermined characteristics such as, for example, temperatures, ionic strength, concentrations and ratios, brought into contact with one another) and formed in situ within the balloon 206—which is itself disposed within the post-surgical cavity. Once the implant 208 has at least partially polymerized or otherwise solidified, the containment structure (such as the balloon 206) may then be opened or otherwise compromised. For example, the balloon 206 may be ruptured by a sharp instrument such as shown at 210, thereby enabling the ruptured and now-deflated balloon 206 to be slipped over the implant 208 and extracted from the cavity through the cavity access path. This leaves the just formulated and formed implant 208 in place, “in situ” within the cavity. The wound leading to the cavity access path may then be closed, thereby sealing the implant 208 within the post-surgical cavity.

According to embodiments of the present inventions, the implant 208 has no existence outside of the balloon 206 within the post-surgical cavity. Only the constituent materials thereof exist separately prior to their respective introduction into the balloon 206. Therefore, the implant 208 is not inserted or delivered within the cavity, but is formulated (and its resultant characteristics determined) and formed (including caused to assume its shape) within a containment structure that is itself inserted within the post-surgical cavity. In this manner, the containment structure acts as a bio-reactor into which the constituent components of the to-be-formed implant are introduced to formulate and form the implant in situ. The implant, therefore, is both formulated (its composition, structure and Characteristics determined) and formed of its constituent materials within the balloon 206, which is itself within the post-surgical cavity.

The implant 208 may include a gel or a pre-polymer composition (such as, for example, alginate) which, after introduction into the balloon 206, may be treated with a catalyst or cross-linker (e.g., divalent cations of bivalent metals) to initiate and cause polymerization or gelation. The material for the implant 208 may also include a material such as gelatin that is a liquid melt at a first temperature and that is capable of solidification to a non-fluent state by exposure to a comparatively lower physiological temperature within the cavity and to an environment such as body thuds.

The materials used to form the implant 208 in situ may include a first reagent such as a polymer (such as, for example, alginate or gelatin) and a second reagent such as an initiator, catalyst or cross-linker. According to embodiments of the present invention, the polymer may be such that it is susceptible to gelling (becoming a gel) when subjected to certain environmental conditions. For example, the polymer may include a solution containing an effective amount of a bio-compatible and bio-degradable natural polymer, such as alginate. As is known, alginate, extracted from seaweed, is a linear copolymer that may include homopolymeric blocks of (1-4)-linked β-D-mannuronate (M) and its C-5 epimer α-L-guluronate (G) residues, respectively, covalently linked together in different sequences or blocks. The monomers may appear in homopolymeric blocks of consecutive G-residues (G-blocks), consecutive M-residues (M-blocks), alternating M and G-residues (MG-blocks) or randomly organized blocks. The alginate may be dispersed in a solvent (such as an aqueous solution, for example). The amount of alginate dispersed in the aqueous solution may be freely chosen, but for the constraint that the alginate solution should have a sufficiently low coefficient of viscosity so as to be efficiently delivered to the balloon 206 within the cavity. Higher concentrations of alginate within a solution will yield firmer gels. For example, a concentration of alginate in the aqueous solution may be selected within the range of about 0.1% to about 30% by weight. For example, a concentration of alginate in the aqueous solution may be selected within the range of about 0.1% to about 80% (for example) by weight. Concentrations outside of these ranges may also be used. The implant material may include a salt of alginic acid, such as, for example, the sodium salt of alginic acid NaC₆H₇O₆.

As is known, an alginate gel may be characterized as being a part solid and part solution. After gelling, water molecules are physically entrapped by the matrix formed by the alginate material. Alginate gel develops in the presence of a divalent ionic solution that may include, for example, cations such as Ca₂+, Br₂+ or Sr₂+. Here, a calcium salt with good, or limited solubility, or complexed Ca₂+ ions may be mixed with an alginate solution into which the calcium ions are released. The gelling initiator or cross-linker may be or include CaCl₂ and the polymer may be or include a sodium alginate solution (such as, for example, the material sold under the trade name of PRONOVA™ and manufactured by NovaMatrix).

The amounts of the polymer and the cross-linking agent may be freely selected such that the resulting implant occupies substantially all of the volume of the inflated balloon 206 and, consequently, substantially all of the volume of the cavity once the balloon 206 is extracted therefrom. Therefore, the size of the cavity may dictate the relative amounts of the polymer such as alginate and of the cross-ling agent. Stated differently, the relative amounts of the constituent reagents of the implant to be formed in situ may be a function of at least the size of the expanded balloon and/or a function of the volume of the post-surgical cavity. The implant 208 may be formed, for example, of about 0.05 cc to about 400 cc of polymer or polymer solution, such as the alginate solution described above. For example, the implant 208 may be formed of about 0.2 cc to about 4 cc of alginate-containing solution. Only as much cross-linking agent as is necessary to cause at least partial gelation of the alginate or other polymer need be used. For example, the alginate-containing solution may be gelled or cross-linked when bathed in about 0.02 cc to about 600 cc of gelling initiating cross-linker. For example, an amount selected from about 0.1 cc to about 8 cc of cross-linker, such NaCl₂, for example, may be effective to gel the alginate-containing solution. However, it is to be understood that the above ranges are only illustrative and that other ranges are possible, as those of skill in this art may appreciate.

According to embodiments of the present inventions, a delivery system may be provided for introducing an initially fluent material through an opening and into an inflated balloon 206 and then activating the material by exposure to a catalyst or cross-linking agent. As those of skill in this art may appreciate, many different configurations of such a delivery system are possible and fall within the scope of the present inventions. The catalyst (e.g., a cross-linking agent) may be combined with a bio-active material such as a therapeutic agent and/or other polymer solution. The cross-linking agent may have itself been treated, agitated or foamed to exhibit a (even temporary) predetermined porosity that may be characterized by a predetermined pore density and/or predetermined pore architecture. Alternatively, the containment structure (e.g., balloon) within which the cross-linking agent is contained may have a predetermined porosity or may include a layer having such predetermined porosity.

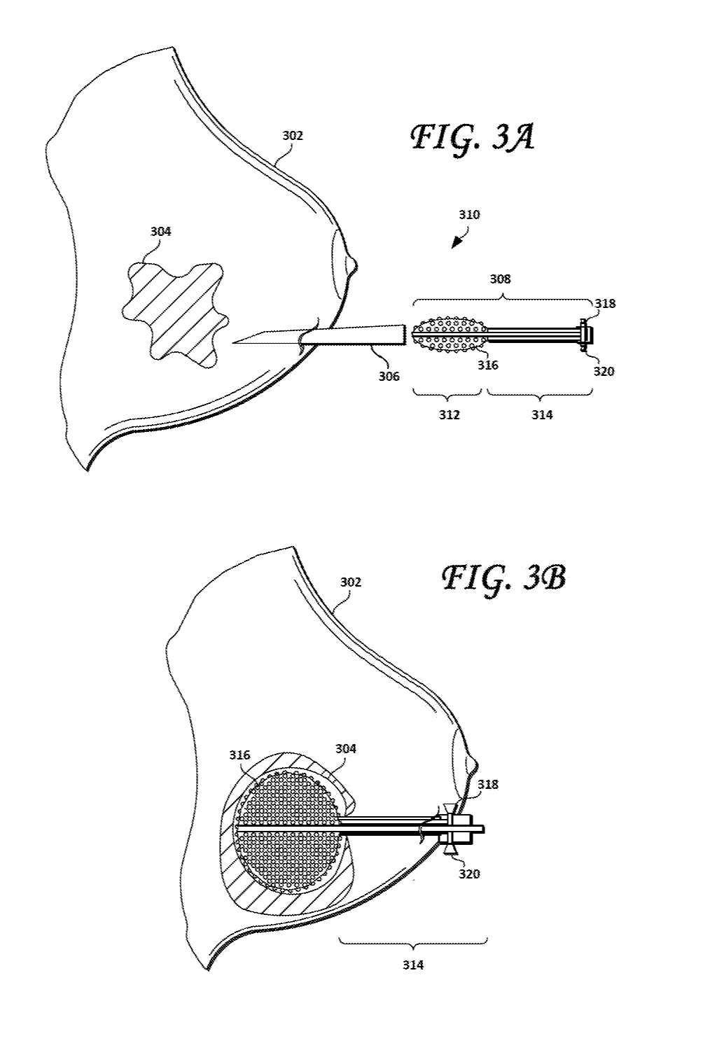

FIG. 3A shows an example of a delivery system 310, a breast 302 into which a post-surgical cavity 304 has been formed. The delivery system 310 may include a trocar introducer 306 and a catheter 308. The distal end of the trocar introducer 306 may be inserted into the post-surgical cavity 304 and the catheter 308 may, in turn, be inserted into the trocar introducer 306 until the distal end thereof is disposed within the post-surgical cavity. This may be done while under guidance from, e.g., an ultrasonic wand such as shown at 204 in FIG. 2B. Alternatively, some other visualizing modality, such as fluoroscopy, may be used to guide the introduction of both the trocar introducer 306 and the catheter 308. FIG. 3A shows the catheter 308 having a first distal portion 312 and a second proximal portion 314. As shown, and according to embodiments of the present inventions, the first distal portion 312 includes a variable geometry containment structure such as an inflatable balloon 316. The second proximal portion 314 may include an elongate section into which are defined and disposed one or more lumens and ports for the delivery (and optionally evacuation of) one or more of the constituent components of the implant to be formed. The lumen or lumens defined within the second proximal portion 314 are in fluid communication with the interior space defined by the sidewalls of the balloon 316. In this manner, a polymer and/or other materials may be delivered from the proximal portion 314 of the catheter 308 into the balloon 316 and/or optionally evacuated therefrom as needed.

According to embodiments of the present inventions, the balloon 316 may be formed of or include, for example, silicone, polyurethane and/or any other suitable bio-compatible material. The balloon, as is known, may be formed (for example) by dipping a preform into a volume of silicone dispersion, coating the external surface(s) of the preform with a layer of silicone, curing the silicone and removing the silicone from the preform. The balloon 316 may, in this manner, have a smooth inner surface. Embodiments of the present inventions may use such a smooth-sided balloon 316. According to such embodiments, the gelling initiator or cross-linker may be introduced within the balloon 316, followed by the alginate solution or other polymer gel such as gelatin. Alternatively, the gelling initiator or cross-linking agent may already be present within the balloon prior to its insertion into the post-surgical cavity 304. The thereafter introduced alginate solution, other polymer gel or gelatin may displace some of the volume of cross-linker already present within the interior of the balloon 316. Such displaced volume of cross-linker may be evacuated through, for example, a port 318, 320 within the proximal portion 314 of the catheter 308. Now bathed in the remaining volume of cross-linker, the alginate solution, other polymer, gel or gelatin begins to solidify through a gradual cross-linking or other solidification process. Alternatively, the cross-linker and the alginate solution, gel or gelatin may be introduced in the opposite order or may be introduced into the interior of the balloon 316 at the same time, taking care that a rapidly solidifying polymer does not clog the delivery lumen(s) of the catheter 308.

While a balloon 316 having a smooth or relatively smooth interior Surface may be used, other embodiments Of the present inventions envisage a porous layer, structure or section disposed, formed on or coupled to the interior surface of the balloon 316. Such a porous layer or discrete section or portion may be of the same or a different material than the material of the balloon 316. For example, both the balloon 316 and the porous layer may be formed of the same material, such as silicone or polyurethane. In that case, a cross-section of the balloon may reveal a porosity gradient from the exterior surface (that surface of the balloon 316 in contact with the cavity sidewalls) to the interior surface of the balloon. Alternatively, the balloon 316 and the porous layer may be formed of different materials. For example, the balloon 316 may be formed of or include silicone, while the porous layer formed on or disposed on the interior surface thereof may be formed of or include polyurethane, or vice-versa. The thickness of the porous layer may be freely chosen according to, for example, the dimensions of the balloon, the concentration of the gelling initiator or cross-linking agent to be contained therein, the amount of polymer to be cross-linked, and the pore morphology of the porous layer. For example, the thickness of the porous layer may be less than a millimeter up to several centimeters and may occupy a volume of up to, for example, one quarter or one half of the interior volume of the expanded containment structure or balloon.

According to an embodiment of the present inventions, such a porous layer may be loaded with a predetermined volume of cross-linker and/or other biologically active substance. The presence of the porous layer (or other layer configured to hold a volume of cross-linking agent) is advantageous, as it can hold, within its porous architecture, a volume of cross-linker and optionally a quantity of some other biologically active and beneficial substance such as, for example, an antimicrobial agent, an analgesic agent, a chemotherapy agent, an anti-angiogenesis agent or a steroidal agent, to name but a few of the possibilities.

According to this embodiment of the present inventions, the balloon 316 may be inserted, in its un-inflated state, into the cavity 304. Thereafter, the balloon 316 may be inflated (with air or CO₂, for example) or otherwise expanded and a volume of cross-linker and optionally some biologically active substance, thereby bathing the interior of the balloon in the resulting solution. The CO₂ inflation or expansion of the balloon may be omitted. Some of that introduced solution will be absorbed within the spongy matrix of the porous layer on the interior surface of the balloon 316. Excess solution may then be evacuated or left in place, to be displaced by the polymer (such as the previously described alginate solution, gel or gelatin) thereafter introduced to the interior of the balloon 316. This polymer introduced into the interior of the balloon 316 may then exert pressure against the cross-linking agent-containing porous layer, thereby releasing the cross-linking agent, which then comes into contact with the introduced polymer (e.g., alginate solution). As the cross-linking agent is released from the porous layer, the alginate (and/or other polymer) solution becomes more and more cross-linked and gradually solidifies. Depending upon the selected concentration of alginate in the introduced solution and the concentration of cross-linking (or other gelling initiator) agent within the porous layer and the time period during which the two are left in contact with one another, the introduced polymer will solidify more or less rapidly and to a greater or lesser degree. This rate is freely selectable by judiciously selecting the amounts of, ratios and concentrations of the constituent components of the implant to be formed. The solidified (e.g., cross-linked) alginate solution then forms the implant that is to be left in place after the balloon is breached or otherwise opened and extracted from the cavity 304.

FIG. 3B shows the balloon 316 in its expanded or inflated state within the cavity 304. As shown in FIG. 3A, the cavity 304 may be irregularly shaped, even if produced by cutting a surface of revolution from the surrounding tissue, due to the different tissue densities within the breast. When the balloon 316 is introduced within the cavity and inflated (either by a gas such as CO₂ and/or through the introduction of the cross-linking agent or polymer) or otherwise expanded, the exterior surface of the balloon will push against the sidewalls of the cavity and may somewhat expand (and regularize the shape of) the cavity and may provide some measure of hemostasis. FIG. 3B shows an embodiment in which the introduction of a catalyst medium (e.g., a volume of about 0.01 cc to about 600 cc), such as a liquid cross-linking solution or gelling initiator at least partially expands the balloon 316 within the cavity 304 until the balloon has been inflated to fill the cavity 304 and conform (at least partially) to the shape of the cavity 304. If the balloon 316 has been previously expanded, the introduction of the cross-linking agent may displace some of the CO₂ previously used to expand the balloon, which displaced gas may be evacuated through, for example, one of the ports 318, 320 of the second proximal portion 314 of the delivery system. 310. According to embodiments of the present inventions, the balloon 316 may include or be formed of a soft and compliant material that is adapted to conform at least partially to the shape of the cavity. The pliability and conformability of the balloon to the shape of the cavity 304 may also be a function of the thickness (which need not be uniform, as is discussed below) of the balloon 316.

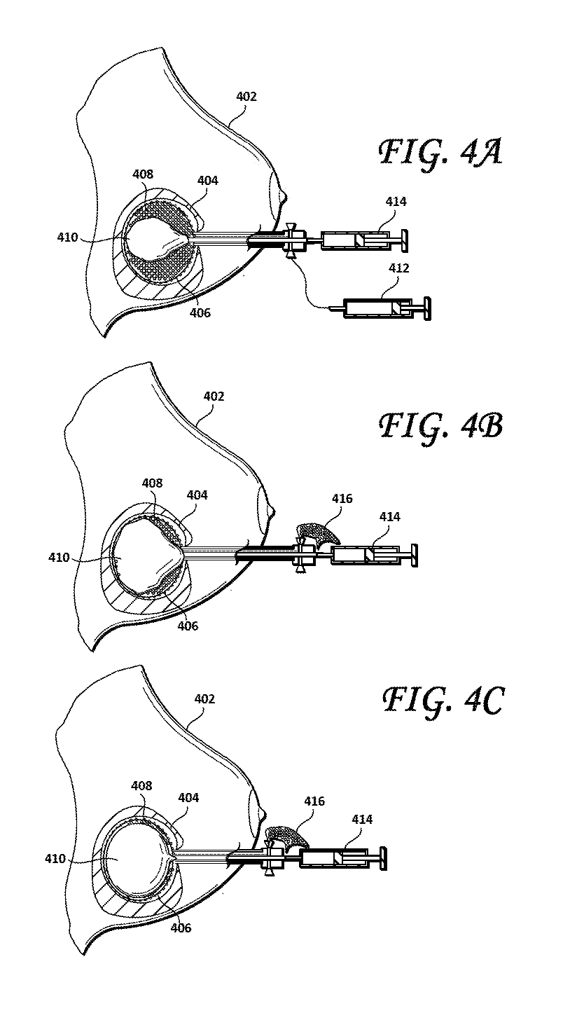

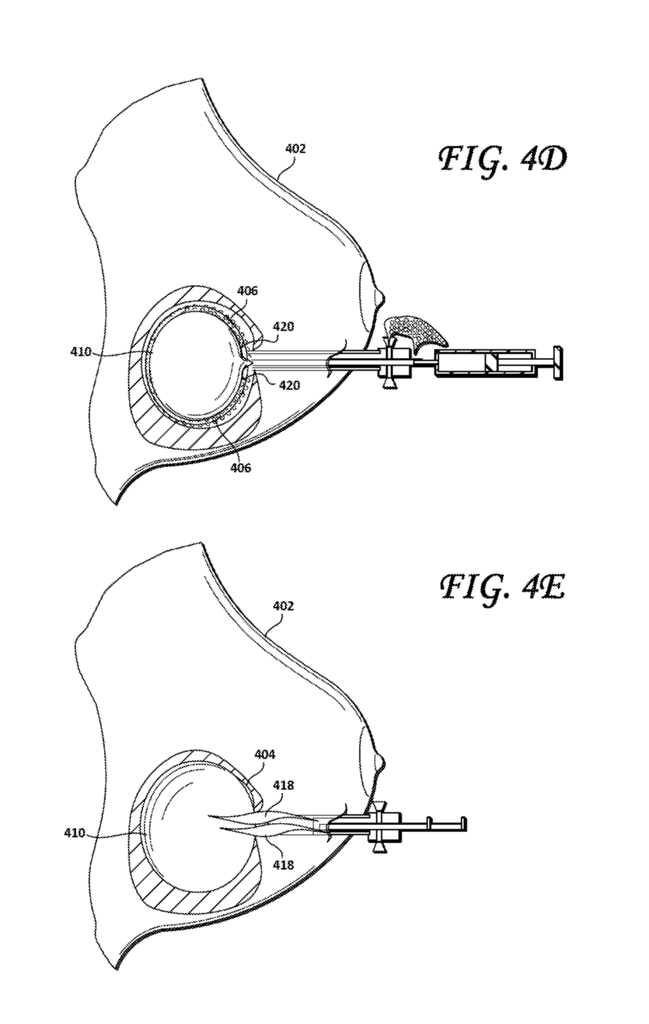

FIG. 4A shows a cavity 404 formed within a breast 402. The balloon 406 of first distal portion of the delivery system is shown in an inflated or expanded state within the cavity 404. A cross-linking solution or other catalyst or gelling initiator is contained with a porous layer(s), section(s) or structure(s) formed on or otherwise coupled to the interior surface of the balloon 406. FIG. 4A shows an embodiment of the present inventions in a state wherein a volume of polymer (an alginate solution, for example) 410 is being introduced within the interior volume of the balloon 406. For example, a volume of about 0.01 cc to about 900 cc of alginate solution may be delivered to the interior of the balloon 406 from the reservoir 414, which interior of the balloon 406 is at least partially filled with crosslinking solution for other gelling initiator or catalyst) from reservoir 412.

FIGS. 4B and 4C show the continued injection or introduction of the polymer (e.g., alginate solution) 410 into the interior volume of the containment structure/balloon 406. The balloon may be fully or partially filled with a cross-linking agent-containing solution and/or may include a layer or layers saturated with the same. As the alginate or other polymer 410 is introduced into the balloon 406, the alginate 410 may gradually displace at least some of the cross-linking agent-containing solution and/or gas or liquid contained therein. The displaced cross-linking agent-containing solution and/or gas or liquid contained therein may be evacuated through one or more of the ports provided in the second proximal portion of the delivery system, as suggested at 416 in FIGS. 4B and 4C, to prevent the balloon 406 from over-expanding within the cavity 404. The just-introduced polymer may then react with the cross-linking solution within which it is now bathed, to form a self-polymerizing solidified or solidifying material within the interior volume of the balloon. Either or both the polymer 410 and/or crosslinking agent-containing solution 408 may also be mixed with or otherwise configured to include other biologically active (such as, for example, therapeutic agents) for dispersion through and/or around the formed solidifying or solidified polymer, to provide additional treatments to the surrounding tissue after the balloon 406 is extracted from the cavity 404.

Once the alginate solution, polymer or gel 410 and the cross-linking agent-containing solution 408 have mixed (or at least have come into contact with one another) and polymerized in situ within the balloon to form a solidified biomaterial (or at least partially solidified material), the containment structure (for example, the balloon 406) may then be opened, breached or otherwise ruptured. The rupturing may be carried out with a sharp object such as a needle, tapered instrument or some other rupturing instrument. Alternatively, the rupturing, may be carried out by some selectively actuable structure(s) on the trocar or the catheter, as suggested at 420 in FIG. 4D. The ruptured balloon 416 (shown in FIG. 4E at 418), which may remain attached to the second proximal portion of the delivery system as shown at 416, may then be withdrawn proximally (e.g., through the trocar) and out from the cavity 404, leaving the just-formed and now resident implant 410 conformed to the surrounding cavity interior, as shown in FIG. 4E. The trocar (if present) may then be removed from the breast tissue, leaving a single small opening for closure.

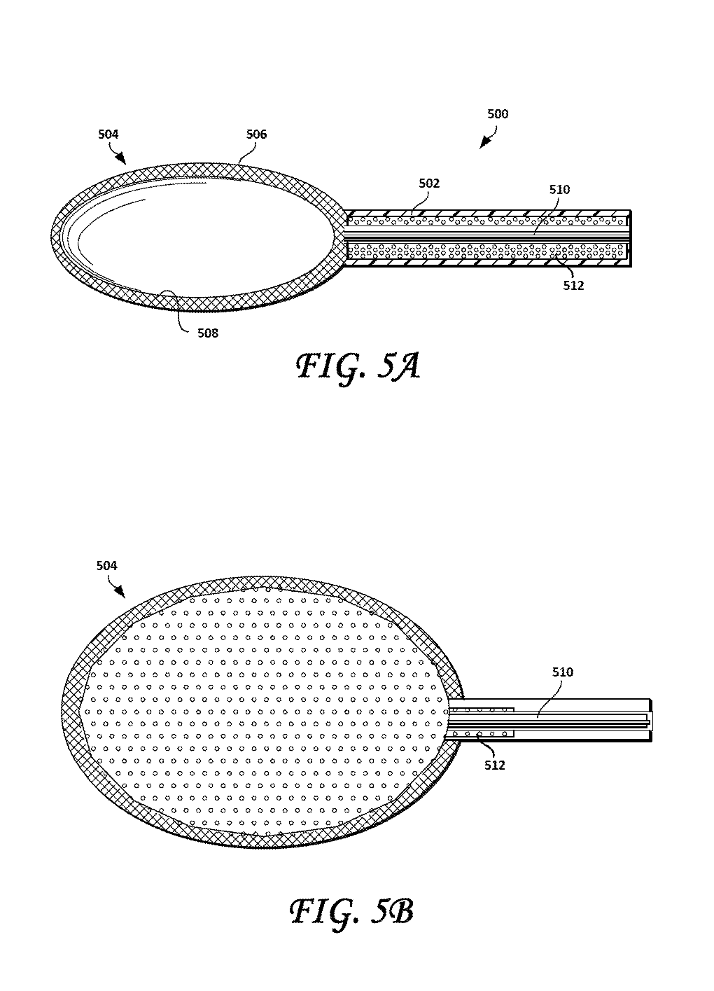

FIG. 5A shows a cross-sectional side view of a delivery system 500, according to an embodiment of the present inventions. As shown, the delivery system 500 includes a shaft 502 that defines a first lumen 510 to deliver gelling initiator or cross-linking agent-containing solution to the interior of the balloon 504 and a second lumen 512 for the delivery of polymer (an alginate solution, for example) to the interior of the balloon 504. The balloon 504 may define an outer surface 506 that is, in use, in contact with the sidewalls of the cavity within the breast and an inner surface that defines the inner volume of the balloon 504. As shown, the inner surface of the balloon 504 may include a porous layer or layers, section(s) and/or structures, as collectively suggested at 508. The porous layer need no overlay the entirety of the inner surface of the balloon 504. FIG. 5B shows a cross-sectional side view of the delivery system 500, in a state in which the balloon has been inflated or otherwise expanded. Such expansion or inflation may be achieved by, for example, injecting a cross-linking agent-containing solution through the lumen 510, as suggested at 510 in FIG. 5B.

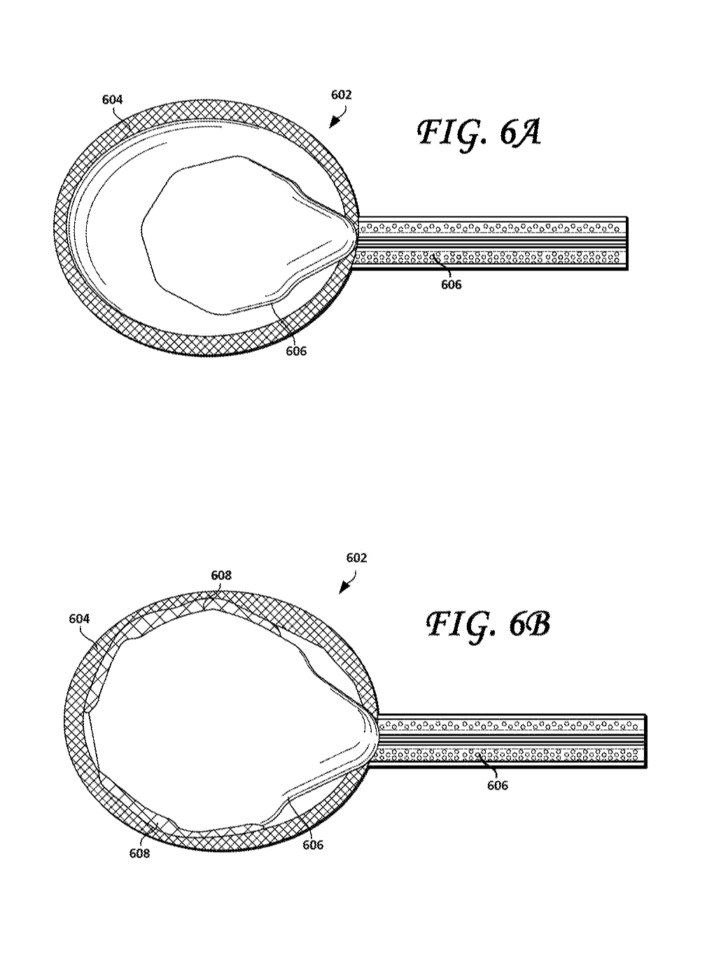

FIG. 6A shows another view of an embodiment of the present inventions. In this view, excess cross-linking agent-containing solution has been drained or otherwise evacuated from the interior Of the balloon 602, leaving a predetermined amount thereof retained within the porous layer(s), section(s) and/or structure(s) 604 within the balloon 602. Evacuating excess cross-linking agent-containing solution may facilitate the introduction of a volume of alginate-containing solution 606 (or other polymer or polymer-containing solution susceptible to selective solidification) into the interior of the balloon, as shown in FIG. 6A. FIG. 6B shows the gradual formation of a solidified (e.g. cross-linked) portions 608 of the introduced alginate (or other polymer-containing solution) at or around the points of contact of the alginate solution with the porous layer 604 within the interior of the balloon 602. At such point(s) of contact, the alginate presses against the porous layer 604 and causes the now-compressed porous layer to release an amount of cross-linking agent-containing solution, which then reacts with the alginate or other polymer by causing a cross-linking or solidification of the alginate at or near the points of contact. Gradually, over time, all or substantially all of the introduced polymer will react with the retained cross-linking agent-containing solution, catalyst or other gelling initiator, to cause the gradual formation, over time, of the implant that is to remain within the cavity in the breast. Either or both of the cross-linking agent-containing solution and the introduced polymer may be agitated, or otherwise caused to assume, at least temporarily, a porous or foam-like appearance, to further facilitate and speed up the cross-linking or gelling process within the balloon 602.

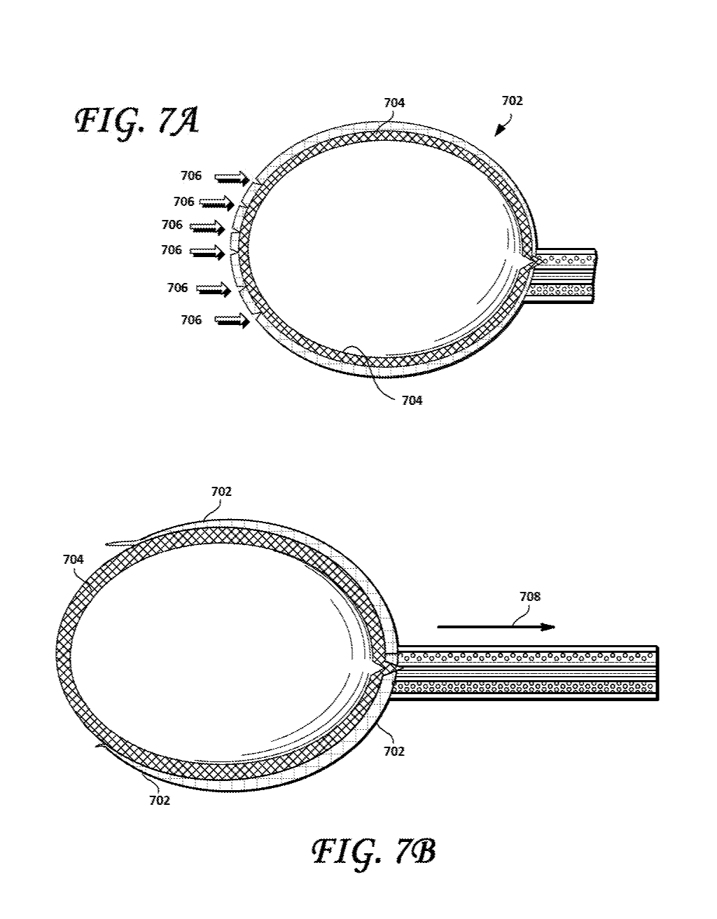

FIG. 7A shows further aspects of embodiments of the present inventions. As shown, the delivery system includes a balloon 702 within a cavity, within which balloon 702 an implant has formed or within which an implant is in the process of forming. To facilitate the rupture of the balloon 702 to enable the easy extraction thereof, the balloon 702 may be formed in such a manner as to define or include one or more locally weaker portions. Such portions may be made weaker by, for example, varying the thickness of the balloon material such that the portions of the balloon to be made weaker are made relatively thinner than the remaining portions of the balloon 702. For example, such locally thinner portions may take the form of notches, striations or weakened sections 706 formed or defined near or at a distal end of the balloon 702. Alternatively, the balloon 702 may be configured to have its distal portion (for example), formed thinner than the relatively thicker proximal portion thereof. In use, when such a balloon is over-inflated, the over-inflated balloon may rupture first at its relatively thinner distal portion. This facilitates the removal of the balloon from the cavity and the deployment of the implant previously contained therein in the body cavity.

Indeed, as shown in FIG. 7B, the balloon 702 has been ruptured by, for example, pulling thereon in the proximal direction (see arrow 708). Pulling on the delivery system (and, therefore, on the balloon 702 within the cavity) stretches the weakened portions (e.g., the notched or other locally thinner portions) of the balloon 702 to and beyond their elastic limits, leading to those portions tearing and giving way. The ruptured balloon 702 may then slip over the cross-linked (e.g., solidified) outer layer 704 of the fully formed or still forming implant.

According to further embodiments, a bio-compatible marker may be introduced along with, for example, the cross-linking agent-containing solution or along with a polymer or a gel. The marker is preferably radio-opaque and echogenic, that is, visible under X-ray and/or ultrasound, for example. The marker may be made from a non-magnetic material, so as to be MRI-compatible. For example, the radio-opaque element may be formed of a bio-compatible metal such as, for example, stainless steel, or titanium, or Nitinol®, a nickel-titanium alloy. The formulated implant will then solidify around the marker, which marker should remain in place even after the cross-linked alginate implant has been resorbed by the body. The presence of such a marker will facilitate the localization of the surgery and subsequent implant formation.

The polymer (such as the alginate-containing solution) and the cross-linking agent-containing solution or cross-linking agent-containing solution mixed or otherwise coupled with a biologically active substance may be provided and packaged separately in pre-measured quantities, with the physician deciding the quantities of each to introduce into the balloon before or during the implant-forming procedure. This and the other embodiments shown and described herein may be provided as an assembled system or provided as a kit, in sterile packaging. The delivery system may be configured for one-time use or portions thereof may be re-usable. Those of skill in this art may recognize other alternative embodiments and all such alternative embodiments are deemed to fall within the scope of the present invention.

Indeed, the disclosed embodiments of the present inventions are not limited to those shown and described herein but may include any number of other variations. Modification of the above-described methods and devices for carrying out the described embodiments are possible and all such modification are deemed to fall within the scope of the present inventions.

1-26. (canceled)

27. A delivery system, comprising:
a first source of polymer:
a second source, separate from the first source, of a gelling initiator that is configured to gel the polymer, and
a catheter configured to deliver the polymer and the gelling initiator to a cavity within the patient such that the delivered polymer and gelling initiator are initially isolated from the cavity and only selectively exposed to the cavity after the polymer has at least partially gelled.

28. The delivery system of claim 27, wherein the catheter comprises a balloon configured to isolate the delivered polymer and gelling initiator from the cavity.

29. The delivery system of claim 28, wherein the balloon comprises a porous interior surface configured to contain at least a portion of the gelling initiator.

30. The delivery system of claim 29, wherein the porous interior surface is configured to release the contained gelling initiator at least when the introduced polymer applies pressure there against.

31. The delivery system of claim 27, wherein the polymer comprises alginate.

32. The delivery system of claim 27, wherein the gelling initiator comprises divalent cations.

33. The delivery system of claim 27, wherein the gelling initiator comprises a cross-linking agent.

34. The delivery system of claim 27, wherein the catheter is configured to deliver the polymer and the gelling initiator separately to the cavity and to isolate the delivered polymer and gelling initiator from the cavity until the polymer has at least partially gelled.

35. The delivery system of claim 28, wherein the balloon comprises locally weaker portions.

36. The delivery system of claim 28, further comprising a marker configured to be delivered within the balloon within the cavity.

37. A method of forming a surgical implant, comprising:
providing a first source of polymer;
providing a second source, separate from the first source, of a gelling initiator that is configured to gel the polymer, and
delivering the polymer and the gelling initiator within a cavity within the patient such that the delivered polymer and gelling initiator are initially isolated from the cavity and only selectively exposed to the cavity after the polymer has at least partially gelled.

38. The method of claim 37, wherein delivering comprises providing a balloon within the cavity, and wherein the method further comprises delivering at least the gelling initiator within an interior space of the balloon that is isolated from the cavity.

39. The method of claim 38, wherein providing the balloon is carried out with the balloon comprising a porous interior surface configured to contain at least a portion of the gelling initiator.

40. The method of claim 39, wherein providing the balloon is carried out with the porous interior surface being configured to release the contained gelling initiator at least When the introduced polymer applies pressure there against.

41. The method of claim 37, wherein the polymer comprises alginate.

42. The method of claim 37, wherein the gelling initiator comprises divalent cations.

43. The method of claim 37, wherein the gelling initiator includes a cross-linking agent.

44. The method of claim 37, wherein delivering comprises delivering the polymer and the gelling initiator separately to the cavity and isolating the delivered polymer and the gelling initiator from the cavity until the polymer has at least partially gelled.

45. The method of claim 38, wherein the balloon includes locally weaker portions.

46. The method of claim 38, further comprising delivering a marker within the balloon within the cavity.
